(19) United States
(12) Patent Application Publication
(10) Pub. No.: US 2019/0180881 A1
(43) Pub. Date: Jun. 13, 2019

(54) MULTIPLE Z-SCORE-BASED NON-INVASIVE PRENATAL TESTING METHOD AND APPARATUS

(71) Applicant: EONEDIAGNOMICS CO., LTD, Incheon (KR)

(72) Inventors: Min Seob LEE, Incheon (KR); Shang Cheol SHIN, Gyeonggi-do (KR); Sung Hoon LEE, Incheon (KR); Hyuk Jung KWON, Gyeonggi-do (KR)

(73) Assignee: EONE DIAGNOMICS GENOME CENTER CO., LTD, Incheon (KR)

(21) Appl. No.: 16/312,999

(22) PCT Filed: Jun. 9, 2017
(86) PCT No.: PCT/KR2017/006048

§ 371 Date: Dec. 21, 2018

(30) Foreign Application Priority Data
Jun. 10, 2016 (KR) 10-2016-0072443

(51) Int. Cl. G16H 50/30; C12Q 1/6869; C12Q 1/6827; G16B 20/20; G16B 40/20; G16H 50/20; G06F 17/18

(52) U.S. Cl. CPC G16H 50/30; C12Q 1/6869; C12Q 1/6827; G06F 17/18; G16B 40/20; G16H 50/20; G16B 20/20

(57) ABSTRACT

The present invention relates to a non-invasive prenatal testing method and, more particularly, to a method for enhancing the sensitivity and accuracy of non-invasive prenatal testing by applying multi-dimensional threshold values based on multiple Z-scores. Designed to reduce false-positive and false-negative possibility by applying two or more Z-score threshold values to aneuploidy detection for one chromosome, the non-invasive prenatal testing method according to the present invention exhibits the effect of obtaining a more sensitive and more accurate test result. Further, the method can minimize test errors in spite of using a small number of nucleotide sequence fragments, with the resultant effect of reducing an experiment cost and thus expensive testing cost and rapidly performing testing with a low expense.

### TECHNICAL FIELD

The present invention relates to a multiple Z-score-based non-invasive prenatal testing method and apparatus, and more specifically, to a method for enhancing the sensitivity and accuracy of non-invasive prenatal testing by applying multi-dimensional threshold values based on multiple Z-scores.

### BACKGROUND ART

One of the important efforts in human medical research lies in the discovery of genetic deformities in the center of adverse health outcomes. Prenatal testing is a process of determining and diagnosing pre-natal fetal diseases, and it mainly identifies fetal chromosomal aneuploidy.

Generally, mothers who are 35 years of age or older; those who themselves or their immediate family members have a history of genetic disorder or congenital anomalies, or those who have multiple pregnancies are classified as high-risk mothers. The main reason for the continued increase of high-risk mothers is due to the increase of the average age of childbirth. When classified as such high-risk mothers, much attention is required for these high-risk mothers for the safety of mothers and fetuses, and it is necessary that these high-risk mothers receive prenatal testing.

Prenatal testing is largely divided into an invasive prenatal testing method and a non-invasive prenatal testing (NIPT) method. The invasive prenatal testing includes amniocentesis, chorionic villi sampling, cordocentesis, etc., but these invasive prenatal testing methods may cause a shock to fetuses during the examination processes thereby inducing miscarriage, illness, or deformity. Accordingly, non-invasive prenatal testing methods are being developed to overcome these problems.

In particular, with the introduction of the next generation sequencing (NGS) technique and the discovery of massively parallel signature sequencing (MPSS), cell-free fetal DNA (cffDNA) in cell-free DNA (cfDNA) in the maternal blood, a non-invasive prenatal testing method utilizing the same was developed.

Since the conventional non-invasive prenatal testing method employs a method in which one Z-score is calculated per chromosome through a normalization process, in which the number of nucleotide sequence fragments (reads) on each chromosome is divided by the number of entire nucleotide sequence fragments, there are sections that are difficult to distinguish between normal chromosomes and aneuploid chromosomes. Therefore, the conventional method has a problem in that errors occur in the testing results.

In addition, since the amount of cell-free fetal DNA present in maternal blood is relatively small, a method of determination by producing a large number of nucleotide sequence fragments has been used. The generation of a large number of nucleotide sequence fragments has an advantage in that errors can be reduced in determining chromosomal aneuploidy, but it has a problem in that the experimental cost is increased thus increasing the test cost.

Since diagnostic errors of fetal anomalies (false positives (FP) and false negatives (FN)) can cause serious consequences, it is important to develop more sensitive and accurate analysis algorithms in non-invasive prenatal testing methods. For the diagnosis of more accurate fetal chromosomal aneuploidy, there is a need for the development of an algorithm that enables a sensitive and accurate determination even for a small number of nucleotide sequence fragments.

### DISCLOSURE

### Technical Problem

To solve the problems in the conventional techniques described above, an object of the present invention is to provide a non-invasive prenatal testing method based on multiple Z-scores for enhancing the sensitivity and accuracy in the diagnosis of fetal chromosomal aneuploidy in the non-invasive prenatal testing method based on the next generation sequencing (NGS) technique using cell-free DNA of maternal blood, in which threshold values are determined and applied by calculating two or more multiple Z-scores per one chromosome, and sensitive and accurate determination is possible with a small number of nucleotide sequence fragments.

Another object of the present invention is to provide an apparatus for performing a non-invasive prenatal testing method based on multiple Z-scores by the present invention.

### Technical Solution

The present invention is to provide a method for providing information for non-invasive prenatal testing, in which the method is a non-invasive prenatal testing method based on multiple Z-scores, including:

(i) extracting cell-free DNA from maternal blood to produce nucleotide sequence fragments of a specimen using a massively parallel sequencing method, which is a next generation sequencing analysis technology;

(ii) comparing the produced nucleotide sequence fragments with the human reference genome sequence, and arranging them in homologous positions thereon;

(iii) calculating the number of the nucleotide sequence fragments arranged for each of the 23 pairs of chromosomes comprising the autosomal and sex chromosomes;

(iv) correcting the number of the nucleotide sequence fragments arranged for each of the 23 pairs of chromosomes by generating a normalized two-dimensional matrix by dividing the number of nucleotide sequence fragments arranged on each chromosome by the number of nucleotide sequence fragments arranged on each the other chromosome;

(v) through the sample obtained in the control group with normal chromosomes, generating multiple normalized two-dimensional matrices by dividing the number of nucleotide sequence fragments arranged on each chromosome by the number of nucleotide sequence fragments arranged on each the other chromosome, and calculating a two-dimensional matrix of a mean value and a two-dimensional matrix of a standard deviation value for each chromosome, using the multiple normalized two-dimensional matrices of the control group;

(vi) calculating multiple Z-scores per each chromosome, using the calculated two-dimensional matrix of a mean value and the two-dimensional matrix of a standard deviation value of the control group, which were obtained in step (v), and the normalized two-dimensional matrix of a specimen obtained in step (iv); and

(vii) determining whether the multiple Z-scores, which were calculated by the other chromosome with respect to the chromosome of a specimen to be observed, pass threshold value of the aneuploidy.

In the non-invasive prenatal testing method based on multiple Z-scores by the present invention, in step (i), the number of the nucleotide sequence fragments of the specimen is in a range of 1 million to 10 million.

In the non-invasive prenatal testing method based on multiple Z-scores by the present invention, the methods in steps (i), ii), and iii) are widely known and used, but it is preferred that the method is performed by a method described below.

About 10 mL of blood is collected from the mother into a Vangenes Cell Free DNA (Vangenes) container and centrifuged (1,900 g, 15 min, room temperature). The separated plasma is transferred into a 1.5 mL container and centrifuged (16,000 g, 15 minutes, room temperature). According to the instructions of the manufacturer (Qiagen), cell-free DNA is isolated from 2 mL of the plasma using the QIAsymphony DSP Virus/Pathogen Midi Kit. According to the instructions of the manufacturer (Life Technology), ion proton sequencing libraries are prepared using the cell-free DNA sample (<100 ng), and nucleotide sequence fragments are produced using the Ion PI™ Chip kit v3.

In the non-invasive prenatal testing method based on multiple Z-scores by the present invention, the produced nucleotide sequence fragments are arranged in the homologous positions of the human reference genome sequence (hg19) using the BWA (version 0.7.10), and the overlapping nucleotide sequence fragments are removed using the Picard (version 1.81), and the number of the nucleotide sequence fragments arranged on each chromosome is calculated using the SAMtools (version 0.1.18).

In the non-invasive prenatal testing method based on multiple Z-scores by the present invention, step (v) consists of: generating multiple normalized two-dimensional matrices by dividing the number of the nucleotide sequence fragments, arranged on each chromosome through the sample in the control group with normal chromosomes, by the number of nucleotide sequence fragments arranged on each different chromosome; and calculating a two-dimensional matrix of a mean value and a two-dimensional matrix of a standard deviation value for each chromosome of the control group, using the multiple normalized two-dimensional matrices.

In the non-invasive prenatal testing method based on multiple Z-scores by the present invention, as illustrated in FIG. 1, step (v) generates multiple normalized two-dimensional matrices (size: 24×24) with respect to 24 chromosomes including autosomal chromosomes (Chromosome Nos. 1 to 22) and sex chromosomes (X, Y). Additionally, a two-dimensional matrix (size: 24×24) of a mean value and a two-dimensional matrix (size: 24×24) of a standard deviation value of each chromosome of the control group are calculated using the multiple normalized two-dimensional matrices (size: 24×24) and generated, respectively.

In the non-invasive prenatal testing method based on multiple Z-scores by the present invention, step (vi) consists of: calculating multiple Z-scores per each chromosome, using the calculated two-dimensional matrix of a mean value and the two-dimensional matrix of a standard deviation value of the control group with normal chromosomes, which were obtained in step (v), and the normalized two-dimensional matrix of a specimen obtained in step (iv).

In particular, the Z-score values are calculated by the following [Equation 1].

$${Zscore}_{i , j} = \frac{{( ratioof \frac{chri}{chrj} )}_{normal} - {mean ( \frac{chri}{chrj} )}_{reference}}{{SD ( \frac{chri}{chrj} )}_{reference}} \qquad [ Equation 1 ]$$

Accordingly, while step (vi) is performed, a total of 24 Z-score values are calculated for each chromosome of the specimen as shown in Table 1 below.

In Equation 1,

$$( ratio of \frac{chri}{chrj} )$$

represents the ratio of the number of nucleotide sequence fragments arranged on each chromosome divided by the number of nucleotide sequence fragments arranged on each different chromosome;

$${mean ( \frac{chri}{chrj} )}_{reference}$$

represents the calculated mean value of the control group having a normal gene obtained in step (v); and

$${SD ( \frac{chri}{chrj} )}_{reference}$$

represents the standard deviation value of the control group having a normal gene.

TABLE 1
Z-scores calculated per each chromosome

| 24 × 24 | chr1 | chr2 | . . . | chr22 | chrX | chrY |
|---|---|---|---|---|---|---|
| chr1 | Z-score | Z-score | . . . | Z-score | Z-score | Z-score |
| | (1, 1) | (1, 2) | | (1, 22) | (1, X) | (1, Y) |
| chr2 | Z-score | Z-score | . . . | Z-score | Z-score | Z-score |
| | (2, 1) | (2, 2) | | (2, 22) | (2, X) | (2, Y) |
| . . . | . . . | . . . | . . . | . . . | . . . | . . . |
| chr22 | Z-score | Z-score | . . . | Z-score | Z-score | Z-score |
| | (22, 1) | (22, 2) | | (22, 22) | (22, X) | (22, Y) |
| chrX | Z-score | Z-score | . . . | Z-score | Z-score | Z-score |
| | (X, 1) | (X, 2) | | (X, 22) | (X, X) | (X, Y) |
| chrY | Z-score | Z-score | . . . | Z-score | Z-score | Z-score |
| | (Y, 1) | (Y, 2) | | (Y, 22) | (Y, X) | (Y, Y) |

In the non-invasive prenatal testing method based on multiple Z-scores by the present invention, in steps (iii) and (v), the arranged nucleotide sequence fragments are divided into sections with a size of 1 to 50 Mb units based on the position of each chromosome and the number of the arranged nucleotide sequence fragments per each section is calculated.

In the non-invasive prenatal testing method based on multiple Z-scores by the present invention, in step (vii), consists of determining whether the multiple Z-scores, which were calculated by each different chromosome with respect to the chromosome of a specimen to be observed, pass the threshold value of aneuploidy sequencially. In particular, in step (vii), it is preferred that the number of the threshold value of the aneuploidy be in a range of 2 to 23, and the normal chromosome specimen are distinguished from the aneuploidy chromosome specimen by applying and determining whether these values pass the threshold value of the aneuploidy sequencially.

In addition, in step (vii), the chromosomes to be observed are 22 pairs of autosomal chromosomes and X and Y sex chromosomes of a fetus, and it is possible to determine at least one chromosome selected from the group consisting of 22 pairs of autosomal chromosomes and X and Y sex chromosomes of the fetus.

In the non-invasive prenatal testing method based on multiple Z-scores by the present invention, in step (vii), the number of the threshold value of the aneuploidy may also be in a range of 2 to 23.

In the non-invasive prenatal testing method based on multiple Z-scores by the present invention, in step (vii), as the chromosomes to be observed, at least one chromosome selected from the group consisting of 22 pairs of autosomal chromosomes and X and Y sex chromosomes of a fetus may be determined.

The present invention also provides a non-invasive prenatal testing apparatus based on multiple Z-scores, which includes:

a production unit, in which cell-free DNA from maternal blood is extracted and nucleotide sequence fragments of a specimen is produced using a massively parallel sequencing method, which is a next generation sequencing analysis technology;

an arranging unit, in which the produced nucleotide sequence fragments are compared with the human reference genome sequence and arranged in homologous positions thereon;

a first calculation unit, in which the number of the nucleotide sequence fragments arranged is calculated for each of the 23 pairs of chromosomes comprising the autosomal and sex chromosomes;

a correction unit, in which a correction is performed by generating a normalized two-dimensional matrix by dividing the number of nucleotide sequence fragments arranged on each chromosome by the number of nucleotide sequence fragments arranged on each different chromosome;

a second calculation unit, in which, through the sample obtained in the control group with normal chromosomes, multiple normalized two-dimensional matrices are generated by dividing the number of nucleotide sequence fragments arranged on each chromosome by the number of nucleotide sequence fragments arranged on each different chromosome, and a two-dimensional matrix of a mean value and a two-dimensional matrix of a standard deviation value for each chromosome with normal chromosomes are calculated, using the multiple two-dimensional matrices of the control group with normal chromosomes;

a third calculation unit, in which multiple Z-scores per each chromosome are calculated using the calculated two-dimensional matrix of a mean value, the two-dimensional matrix of a standard deviation value of the control group, and the normalized two-dimensional matrix of a specimen obtained; and

a determination unit, in which it is determined whether the multiple Z-scores, which were calculated by each different chromosome with respect to the chromosome of a specimen to be observed, sequentially pass the threshold value of the aneuploidy.

### Advantageous Effects

The non-invasive prenatal testing method by the present invention has an effect that more sensitive and accurate test results can be obtained by reducing the possibilities of false-positive and false-negative by applying two or more Z-score threshold values for the test of aneuploidy of one chromosome.

Additionally, since the non-invasive prenatal testing method by the present invention can minimize testing errors despite the use of a small number of nucleotide sequence fragments, the method of the present invention has an effect being capable of rapidly performing the test even with a low expense by reducing the high-cost test due to the reduced experimental cost.

Additionally, since sections are divided into units with a certain size based on the positions on each chromosome, and the number of nucleotide sequence fragments arranged per each section is calculated, it is possible to confirm, on which area of each chromosome, the partial amplification and deletion occur, and additionally, the method has an effect of being able to more accurately confirm the patter of chromosomal aneuploidy.

### DESCRIPTION OF DRAWINGS

FIG. 1 is a schematic diagram illustrating the process of normalizing the number of arranged nucleotide sequence fragments according to an embodiment of the present invention.

FIG. 2 is a scatter plot illustrating the accuracy of the non-invasive prenatal testing (NIPT) in a small number of nucleotide sequence fragments according to an embodiment of the present invention.

FIG. 3 is a scatter plot illustrating the analysis results of 3 million nucleotide sequence fragments, which were randomly extracted according to an embodiment of the present invention.

FIG. 4 is a scatter plot illustrating the analysis results of 1 million nucleotide sequence fragments, which were randomly extracted according to an embodiment of the present invention.

FIG. 5A is a schematic diagram illustrating a two-dimensional matrix of an aneuploidy chromosome specimen according to an embodiment of the present invention.

FIG. 5B is a schematic diagram illustrating a two-dimensional matrix of an aneuploidy chromosome specimen according to another embodiment of the present invention.

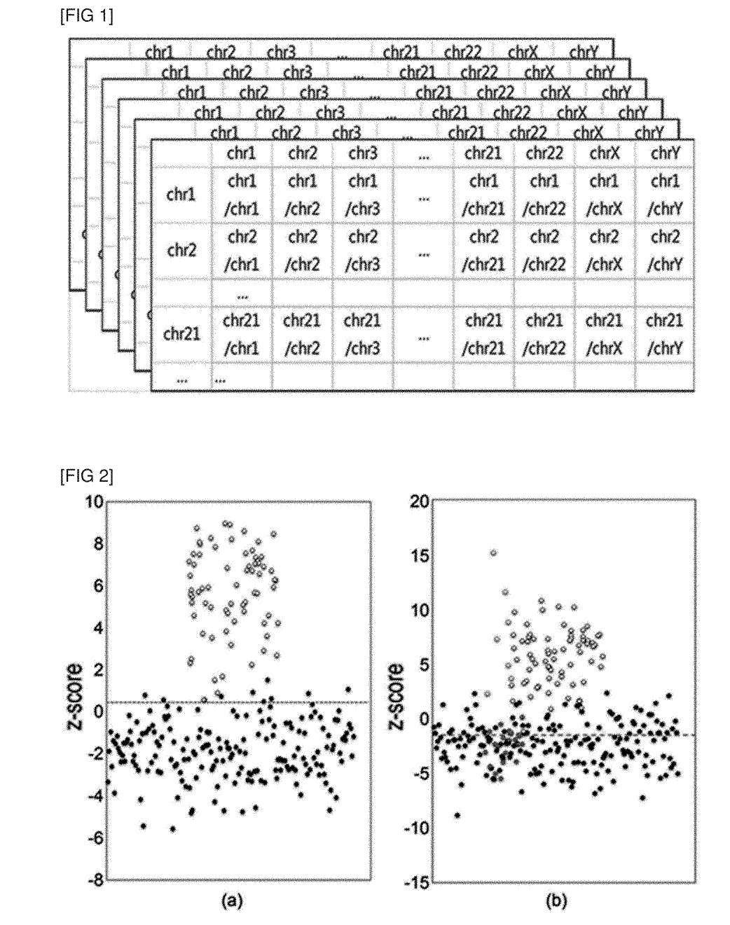

### MODE FOR INVENTION

Hereinafter, the present invention will be described in detail with reference to examples. However, the present invention is not limited by the following examples.

### <Experimental Example 1> Production of Nucleotide Sequence Fragments

Specimens were collected from 216 mothers, in which 7 specimens were those with the aneuploidy chromosome of trisomy 21.

From each maternal blood of the 216 mothers, cell-free DNA was extracted from the 216 specimens by performing: extracting cell-free DNA from maternal blood to produce nucleotide sequence fragments of a subject using a massively parallel sequencing method, which is a next generation sequencing analysis technology; comparing the produced nucleotide sequence fragments with the human reference genome sequence, and arranging them in homologous positions thereon; and calculating the number of the nucleotide sequence fragments arranged for each of the 23 pairs of chromosomes comprising the autosomal and sex chromosomes; and at least 7 million nucleotide sequence fragments were produced therefrom.

About 10 mL of blood was collected from the mother into a Vangenes Cell Free DNA (Vangenes) container and centrifuged (1,900 g, 15 min, room temperature). The separated plasma was transferred into a 1.5 mL container and centrifuged (16,000 g, 15 minutes, room temperature). According to the instructions of the manufacturer (Qiagen), cell-free DNA was isolated from 2 mL of the plasma using the QIAsymphony DSP Virus/Pathogen Midi Kit. According to the instructions of the manufacturer (Life Technology), ion proton sequencing libraries were prepared using the cell-free DNA sample (<100 ng), and 3 million nucleotide sequence fragment sets and 1 million nucleotide sequence fragment sets were randomly produced from the 7 million nucleotide sequence fragments per specimen, using the Ion PI™ Chip kit v3.

### <Comparative Example> Non-Invasive Prenatal Testing Method Using Only One Z-Score

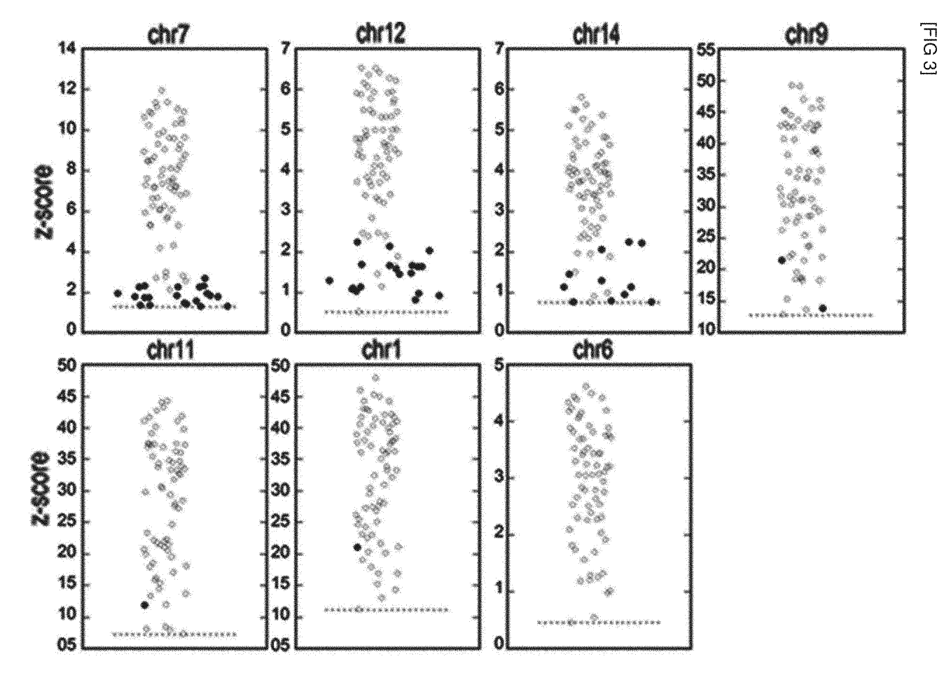

An analysis was performed with regard to the conventional non-invasive prenatal testing method, where only one Z-score is used, using the randomly extracted 3 million nucleotide sequence fragment sets and the 1 million nucleotide sequence fragment sets, and the results are shown in FIG. 2.

Each dot shown in FIG. 2 is as follows.

Black dot: Z-score for normal chromosome specimens randomly extracted from produced nucleotide sequence fragments

white dot: Z-score for aneuploidy chromosome specimens (Trisomy 21) randomly extracted from produced nucleotide sequence fragments

Red dot: Z-score for normal chromosome specimens obtained from produced nucleotide sequence fragments

Red bordered dot: Z-score for aneuploidy chromosome specimens (Trisomy 21) obtained from produced nucleotide sequence fragments

Red dotted line: the lowest value among the Z-scores for aneuploidy chromosome specimens, which is a threshold value used for aneuploidy detection

As illustrated in FIG. 2, when Z-scores that can distinguish all of the Trisomy 21 specimens were used, 9 specimens with false positive were discovered in the analysis where 3 million nucleotide sequence fragments were used by the embodiment of the present invention (FIG. 2A), and 52 specimens with false positive were discovered in the analysis where 1 million nucleotide sequence fragments were used by the embodiment of the present invention (FIG. 2B).

As can be seen in FIG. 2, the analysis, where the 3 million nucleotide sequence fragments were used by the non-invasive prenatal testing method by Comparative Example, 9 normal chromosome specimens among the 216 specimens were mistakenly determined as aneuploidy chromosomes thus showing 95.1% of specificity, whereas the analysis, where the 1 million nucleotide sequence fragments were used, 52 normal chromosome specimens among the 216 specimens were mistakenly determined as aneuploidy chromosomes thus showing a low specificity of 75.1%.

### <Example 1> Non-Invasive Prenatal Testing Method Using Multiple Z-Scores

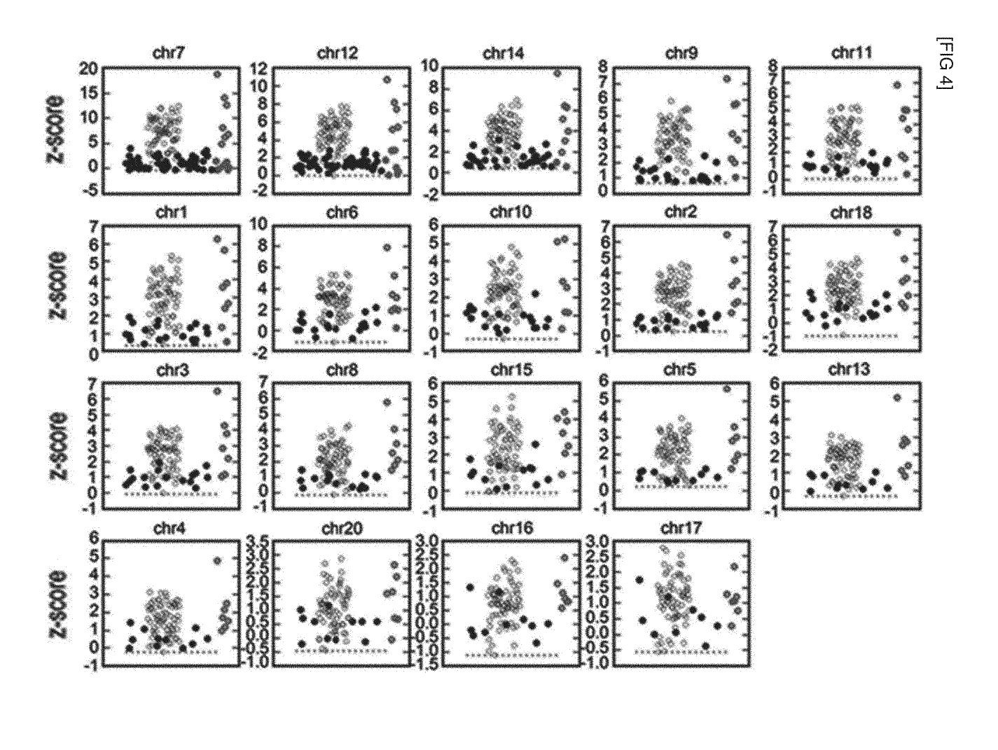

An analysis was performed with regard to the 3 million nucleotide sequence fragment sets, which were randomly extracted from the nucleotide sequence fragments produced in Experimental Example 1, using multiple Z-scores by embodiments of the present invention, and the results are shown in FIG. 3.

Each dot shown in FIG. 3 is as follows.

Black dot: Z-score for normal chromosome specimens randomly extracted from produced nucleotide sequence fragments

white dot: Z-score for aneuploidy chromosome specimens (Trisomy 21) randomly extracted from produced nucleotide sequence fragments

Red dotted line: the lowest value among the Z-scores for aneuploidy chromosome specimens, which is a threshold value used for aneuploidy detection

As illustrated in FIG. 3, the specimens consist of 187 normal chromosome specimens and 70 aneuploid chromosome specimens (Trisomy 21).

In particular, it was determined whether passed as the aneuploidy threshold value by applying the 7 Z-scores with regard to the chromosome nos. 7, 12, 14, 9, 11, 1, and 6 among the 23 Z-scores, with regard to the chromosome no. 21 (chr21), sequentially and, as a result, normal chromosome specimens and aneuploidy chromosome specimens were distinguished with 100% sensitivity and 100% specificity.

### Example 2

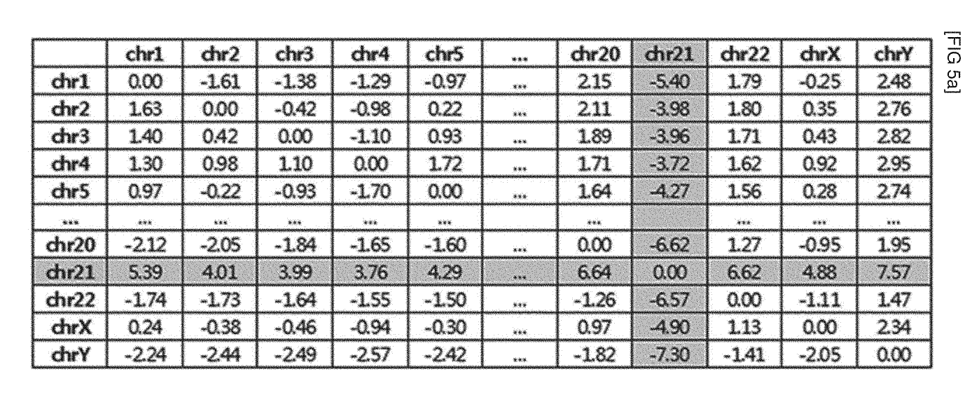

An analysis was performed with regard to the 1 million nucleotide sequence fragment sets of specimens, which were produced in Experimental Example 1, by the method of Example 1 according to the present invention, and the results are shown in FIG. 4.

Each dot shown in FIG. 4 is as follows.

Black dot: Z-score for normal chromosome specimens randomly extracted from produced nucleotide sequence fragments

White dot with Black border: Z-score for aneuploidy chromosome specimens (Trisomy 21) randomly extracted from produced nucleotide sequence fragments

Red dot: Z-score for normal chromosome specimens obtained from produced nucleotide sequence fragments

White dot with Red border: Z-score for aneuploidy chromosome specimens (Trisomy 21) obtained from produced nucleotide sequence fragments

Red dotted line: the lowest value among the Z-scores for aneuploidy chromosome specimens, which is a threshold value used for aneuploidy detection

As illustrated in FIG. 4, the specimens consist of 209 normal chromosome specimens and 7 aneuploid chromosome specimens (Trisomy 21).

In particular, among the 23 Z-scores with regard to the chromosome no. 21 (chr21), it was determined whether passed the aneuploidy threshold value by applying the 19 Z-scores calculated with regard to the chromosome nos. 7, 12, 14, 9, 11, 1, and 6, and the chromosome nos. 10, 2, 18, 3, 8, 15, 5, 13, 4, 20, 16, and 17, sequentially.

As a result, normal chromosome specimens and aneuploidy chromosome specimens were distinguished with 100% sensitivity and 95.6% specificity.

The results with regard to the 1 million data and 3 million data in Comparative Example and Examples 1 and 2 were compared, and the results are shown in Table 2 below.

TABLE 2
Comparison of sensitivity and specificity between the conventional
NIPT method and NIPT method by the present invention

| | | | | | | | |
|---|---|---|---|---|---|---|---|
| | | | | | | Sensi- | Speci- |
| | | | | | | tivity | ficity |
| Method | #Sample | #TP | #FP | #TN | #FN | (%) | (%) |

Comparative Example

| | | | | | | | |
|---|---|---|---|---|---|---|---|
| 3M-reads | 187 | N/A | 9 | 178 | N/A | N/A | 95.1 |
| 1M-reads | 216 | 7 | 52 | 157 | 0 | 100 | 75.1 |

NIPT by the present invention

| | | | | | | | |
|---|---|---|---|---|---|---|---|
| Example 1 | 187 | N/A | 0 | 187 | N/A | N/A | 100 |
| 3M-reads | | | | | | | |
| Example 2 | 216 | 7 | 9 | 200 | 0 | 100 | 95.6 |
| 1M-reads | | | | | | | |

TP: True positive;
FP: False positive;
TN: True negative;
FN: False negative

As can be seen in Table 2, in the case of the conventional non-invasive prenatal testing method where one Z score is used by Comparative Example, more accurate results with higher sensitivity and specificity were obtained as the number of the produced nucleotide sequence fragments became greater.

On the contrary, in the case of the non-invasive prenatal testing method based on multiple Z-scores according to the present invention, more excellent and reliable analysis results were obtained although the number of the produced nucleotide sequence fragments was smaller.

In both analyses of the 3 million nucleotide sequence fragment sets and the 1 million nucleotide sequence fragment sets randomly extracted from the produced nucleotide sequence fragments, the non-invasive prenatal testing method based on multiple Z-scores according to the present invention exhibited more excellent specificity compared to the conventional non-invasive prenatal testing method.

### <Experimental Example 2> Calculation of Number of Nucleotide Sequence Fragments by Dividing Sections

In addition to the method of Experimental Example 1, in steps (iii) and (iv), a step of dividing sections into units with a certain size based on each chromosomal position was added, and the number of nucleotide sequence fragments arranged per section was calculated. In particular, the unit of with a certain size is preferred to be in a range of 1 Mb to 50 Mb.

### <Example 3> Analysis of Aneuploidy Chromosomal Specimen (Trisomy 21)

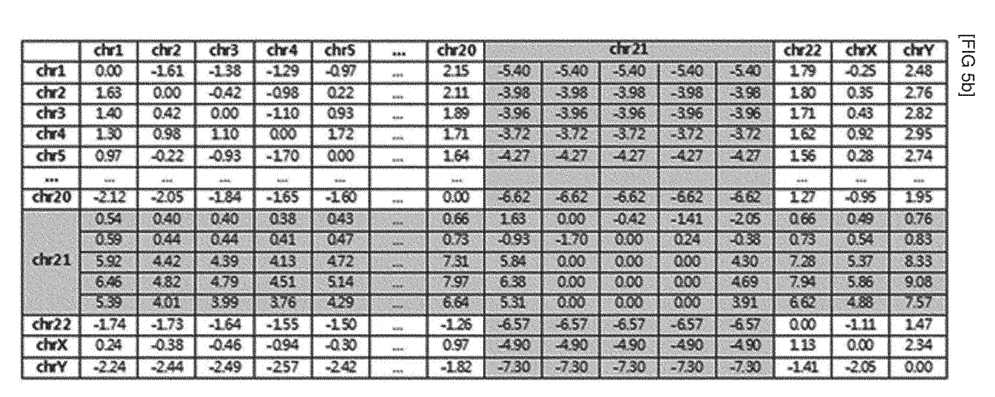

An analysis of the aneuploidy chromosomal specimen (Trisomy 21) was performed for the samples obtained in Experimental Example 1 and Experimental Example 2, and the results are shown in FIG. 5A.

As shown in FIG. 5A, it was confirmed that Z-score values were highly expressed in the chr21 section (crosswise). This indicates that the aneuploidy chromosomal specimen is the Trisomy 21 specimen which has an abnormality on chr21, and one cell of the chr21 section represents each Z-score and it becomes the basis for determining aneuploidy by comparing with the threshold values.

With respect to the Trisomy 21 specimen, analyses were performed by dividing the section into units with a size of 10 Mb, and the analysis results are shown in FIG. 5B.

In particular, reviewing the chr21 section (crosswise), the Z-score value of the short arm section (upper part) of the chromosome was expressed low, whereas the Z-score value of the long arm section (lower part) was expressed high. This indicates that the long arm section of chromosome 21 shows chromosomal aneuploidy while the short arm section does not show chromosomal aneuploidy

Additionally, Example 3 is applicable not only to chromosome 21, but also to the identification of sex chromosomes (e.g., chromosome nos. 9, 13, and 18) and sex chromosomes including X and Y.

Accordingly, when the analysis is performed by dividing the section into units with a certain size by the method of Example 3, it is possible to confirm whether a partial amplification and deletion occurs in which region on each chromosome and a clearer chromosomal aneuploidy pattern can be confirmed.

The embodiments of the present invention are not limited to the embodiments described above. Any embodiment having substantially the same constitution as the technical idea described in the claims of the present invention and achieving the same operational effect should be included in the technical scope of the present invention.

### INDUSTRIAL APPLICABILITY

Designed to reduce false-positive and false-negative possibility by applying two or more Z-score threshold values to aneuploidy detection for one chromosome, the non-invasive prenatal testing method according to the present invention exhibits the effect of obtaining a more sensitive and more accurate test result. Further, the method can minimize test errors despite using a small number of nucleotide sequence fragments, with the resultant effect of reducing an experiment cost and thus expensive testing cost and rapidly performing testing with a low expense. Additionally, sections are divided into units with a certain size based on each chromosomal position, and the number of nucleotide sequence fragments arranged per section is calculated. Therefore, it is possible to confirm whether a partial amplification and deletion occurs in which region on each chromosome and a clearer chromosomal aneuploidy pattern can be confirmed, and thus the present invention is acknowledged to have industrial applicability.

1. A method for providing information for non-invasive prenatal testing, wherein the method is a non-invasive prenatal testing method based on multiple Z-scores, comprising:
(i) extracting cell-free DNA from maternal blood to produce nucleotide sequence fragments of a specimen using a massively parallel sequencing method, which is a next generation sequencing analysis technology;
(ii) comparing the produced nucleotide sequence fragments with the human reference genome sequence, and arranging them in homologous positions thereon;
(iii) calculating the number of the nucleotide sequence fragments arranged for each of the 23 pairs of chromosomes comprising the autosomal and sex chromosomes;
(iv) correcting by generating a normalized two-dimensional matrix by dividing the number of nucleotide sequence fragments arranged on each chromosome by the number of nucleotide sequence fragments arranged on each different chromosome;
(v) through the sample obtained in the control group with normal chromosomes, generating multiple normalized two-dimensional matrices by dividing the number of nucleotide sequence fragments arranged on each chromosome by the number of nucleotide sequence fragments arranged on each different chromosome, and calculating a two-dimensional matrix of a mean value and a two-dimensional matrix of a standard deviation value for each chromosome with normal chromosomes, using the multiple two-dimensional matrices of the control group with normal chromosomes;
(vi) calculating multiple Z-scores per each chromosome, using the calculated two-dimensional matrix of a mean value and the two-dimensional matrix of a standard deviation value of the control group with normal chromosomes, which were obtained in step (v), and the normalized two-dimensional matrix of a specimen obtained in step (iv); and
(vii) determining whether the multiple Z-scores, which were calculated by each different chromosome with respect to the chromosomes of a specimen to be observed, sequentially pass the threshold value of the aneuploidy.

2. The method of claim 1, wherein, in step (i), the number of nucleotide sequence fragments of the specimen is in a range of 1 million to 10 million.

3. The method of claim 1, wherein, in step (vii), the number of the aneuploidy threshold value is in a range of 2 to 23.

4. The method of claim 1, wherein, in step (vii), the chromosomes to be observed comprise at least one chromosome selected from the group consisting of 22 pairs of autosomal chromosomes and X and Y sex chromosomes of a fetus.

5. The method of claim 1, wherein, in step (vii), when the multiple Z-scores sequentially pass the aneuploidy threshold value, the chromosome is determined to be a normal chromosome.

6. The method of claim 1, wherein the arranged nucleotide sequence fragments with respect to the subject specimen of step (iii) and the arranged nucleotide sequence fragments with respect to the control group with normal chromosomes are divided into sections with a size of 1 to 50 Mb units based on the position of each chromosome and the number of the arranged nucleotide sequence fragments per each section is calculated.

7. A non-invasive prenatal testing apparatus for performing the non-invasive prenatal testing method based on multiple Z-scores of claim 1, comprising:
a production unit, in which cell-free DNA from maternal blood is extracted and nucleotide sequence fragments of a specimen is produced using a massively parallel sequencing method, which is a next generation sequencing analysis technology;
an arranging unit, in which the produced nucleotide sequence fragments are compared with the human reference genome sequence and arranged in homologous positions thereon;
a first calculation unit, in which the number of the nucleotide sequence fragments arranged is calculated for each of the 23 pairs of chromosomes comprising the autosomal and sex chromosomes;
a correction unit, in which a correction is performed by generating a normalized two-dimensional matrix by dividing the number of nucleotide sequence fragments arranged on each chromosome by the number of nucleotide sequence fragments arranged on each different chromosome;
a second calculation unit, in which, through the sample obtained in the control group with normal chromosomes, multiple normalized two-dimensional matrices are generated by dividing the number of nucleotide sequence fragments arranged on each chromosome by the number of nucleotide sequence fragments arranged on each different chromosome, and a two-dimensional matrix of a mean value and a two-dimensional matrix of a standard deviation value for each chromosome with normal chromosomes are calculated, using the multiple two-dimensional matrices of the control group with normal chromosomes;
a third calculation unit, in which multiple Z-scores per each chromosome are calculated using the calculated two-dimensional matrix of a mean value and the two-dimensional matrix of a standard deviation value of the control group, and the normalized two-dimensional matrix of a specimen; and
a determination unit, in which it is determined whether the multiple Z-scores, which were calculated by each different chromosome with respect to the chromosome of a specimen to be observed, sequentially pass the threshold value of aneuploidy.
